(19) United States
(12) Patent Application Publication
(10) Pub. No.: US 2025/0256043 A1
(43) Pub. Date: Aug. 14, 2025

(54) METHOD FOR AEROSOL DOSIMETRY PREDICTIONS IN WHOLE LUNG

(71) Applicant: Philip Morris Products S.A., Neuchatel (CH)

(72) Inventors: Arkadiusz KUCZAJ, Duedingen (CH); Francesco LUCCI, Zurich (CH)

(73) Assignee: Philip Morris Products S.A., Neuchatel (CH)

(21) Appl. No.: 18/856,760

(22) PCT Filed: May 24, 2023
(86) PCT No.: PCT/EP2023/063953

§ 371 Date: Oct. 14, 2024

(30) Foreign Application Priority Data
Jun. 3, 2022 (EP) 22177282.5

(51) Int. Cl. A61M 11/04; G16H 20/13; G16H 40/63

(52) U.S. Cl. CPC A61M 11/042; G16H 20/13; G16H 40/63; A61M 2202/0007; A61M 2202/04; A61M 2205/3327; A61M 2205/3334; A61M 2205/3379; A61M 2205/50; A61M 2230/005; A61M 2230/42

(57) ABSTRACT

A system is provided, including: an aerosol-generating device; an inhalation guidance component; and one or more processors configured to capture an aerosol inhalation profile of a user of the device, feed the captured profile to a respiratory system model, generate, based on the model, inhalation guidance instructions, and provide the instructions to the user at the component. A system is also provided, including: an aerosol-generating device; a settings determination component; and one or more processors configured to determine, at the component, one or more settings of the device based on a respiratory system model, and control the device to provide aerosol based on the settings determined by the component. A computer-implemented modular respiratory system model for transient aerosol dosimetry is also provided. A computer-implemented method of generating a modular respiratory system model for transient aerosol dosimetry is also provided.

The present disclosure relates to a model, method, and system for transient aerosol dosimetry. Specifically, the present disclosure relates to generating a modular respiratory system model for determining deposition of inhaled aerosol over time in a region of interest of the respiratory system of a user. In addition, the present disclosure relates to an aerosol generating device using the generated modular respiratory system model to control aerosol deposition in the respiratory system of the user.

Chronic Obstructive Pulmonary Disease (COPD) is estimated to be the third leading cause of death worldwide. COPD results from long-term exposure to harmful gases and particles combined with individual factors. Pulmonary delivery is the first choice for treating respiratory diseases and it is of great interest for other diseases offering several advantages such as faster onset of action and easier non-invasive administration. A major challenge in inhalation toxicity or inhaled drug efficacy assessment is the appropriate and accurate determination of the delivered dose. Medicine dosage can be determined experimentally by using idealized geometries (i.e., bent pipe, Alberta Idealized Throat or NGI from Copley Scientific) or by using 3D printed lung casts extracted from patients' micro-CT scans, as described in CN202010101213. The advantage of micro-CT derived 3D casts is the possibility of analyzing personalized geometries for each patient. However, this technology is limited to the analysis of only the upper respiratory tract and does not allow capturing and reproducing aerosol behavior in the deeper alveolar lung. Therefore, the analysis of aerosol inhalation is compromised. Moreover, experimentally reproducing the correct atmospheric environment inside the lung cast is a technically difficult task. In particular, atmospheric conditions related to humidity at airway surfaces has proved to be a serious challenge. Finally, experiments tend to be expensive and time consuming.

Alternatively, computational modelling has been used to predict dosage, this includes Computational Fluid Dynamic (CFD) models and whole lung dosimetry models such as the Multiple-Path Particle Dosimetry Model (MPPD) and the International Commission on Radiological Protection (ICRP) human respiratory model. While CFD allows for very detailed analysis on realistic geometries extracted from patient scans, it suffers from similar limitations of the experiments in terms of being limited to only the upper airways. Recent attempts to apply CFD to a simplified lung geometry and by assuming a similarity between lung branch generations have allowed for the simulation of a representative full airway tree. But CFD is still very labor intensive, computationally demanding and, in particular, has a very long execution time. Whole lung models, such as MPPD, have been established as the standard for inhaled dose estimations in the toxicology and pharmaceutical field. However, such whole lung models are simplified models with only partial representation of the aerosol physics, which limits their application to simple non-reactive and non-evolving aerosols. In addition, such models are not able to capture the effect of different breathing profiles.

Hence, there is a need to enhance current dosimetry models such that they are able to estimate inhaled aerosol deposition in the respiratory system under realistic conditions and in a computationally efficient manner. It would be desirable that dosimetry models capture all main processes determining the aerosol transport and aerosol deposition over time. Further, it would be desirable that dosimetry models account for different aerosol properties, inhalation topologies, and personalized lung morphologies, while being computationally efficient. Further, it would be desirable that dosimetry models provide instructions to the aerosol generating device and guidance to the user to control aerosol deposition in the respiratory system of the user.

According to an aspect of the present invention, there is provided a system comprising an aerosol generating device and an inhalation guidance unit, the system comprising: one or more processors configured to perform the steps of: capturing an aerosol inhalation profile of a user of the aerosol generating device; feeding the captured aerosol inhalation profile to a respiratory system model; generating, based on the respiratory system model, inhalation guidance instructions; and providing the inhalation guidance instructions to the user at the inhalation guidance unit.

By configuring the system to capture the aerosol inhalation profile of the user, and to feed it back to the respiratory system model, the user of the aerosol generating device is able to interactively adapt the inhalation behavior based on the inhalation guidance instructions generated based on the model and provided at the inhalation guidance unit, thereby enabling the user to reach a desired target aerosol deposition. Capturing the aerosol inhalation profile of the user may be done in real-time. Feeding the captured aerosol inhalation profile to the respiratory system model may be done in real-time. Generating the inhalation guidance instruction and providing the inhalation guidance instructions to the user may be done in real-time. Hence, the user may be able to interactively adapt, in real-time, the inhalation behavior based on the generated inhalation guidance instructions, thereby enabling the user to reach a desired target aerosol deposition, evolution, absorption, and desorption, in real-time.

Capturing the aerosol inhalation profile of the user may comprise determining, in real-time, at least one of an inhalation-exhalation flow rate, a puff rate, and a rate of variation of lungs volume.

Determining, in real-time, the inhalation-exhalation flow rate, puff rate, and the rate of variation of lungs volume, may for example be done using sensors counting inhalation-exhalation cycles, sensors counting aerosol puff cycles, and sensors capturing flow inhalation-exhalation flow profiles.

By capturing the aerosol inhalation profile of the user in real-time, the inhalation behavior of the user may be realistically and quickly adapted to match a desired target aerosol deposition using the inhalation guidance instructions. The inhalation guidance instructions may, for example, indicate to the user a personalized optimal aerosol inhalation profile, an inhalation-exhalation flow rate, or a puff rate, to interactively guide the user to adapt the inhalation behavior in order to reach a desired target aerosol deposition.

The respiratory system model may be a computer-implemented modular respiratory system model for transient aerosol dosimetry, and the model may comprise: a plurality of airway modules, each airway module representing at least a part of an airway generation of the respiratory system; and an inhalation topology module configured for setting an input flow to the respiratory system model over time, wherein the input flow comprises an aerosol inhalation profile, wherein each airway module is configured to determine, based on the input flow set by the inhalation topology module, deposition of the inhaled aerosol over time in the at least a part of the airway generation of the respiratory system represented by the airway module.

By providing a respiratory system model for transient aerosol dosimetry, comprising a plurality of airway modules each of which representing at least a part of an airway generation of the respiratory system, a modular respiratory system model is advantageously implemented, wherein the airway modules can serve as basic building blocks which can be readily and efficiently combined in a modular and computationally efficient manner to quickly model a region of interest of the respiratory system. The region of interest may comprise each airway generation of the respiratory system or any specific part of a lobe of a lung of the respiratory system.

Further, by having an inhalation module configured for setting an input flow, comprising an aerosol inhalation profile, to the respiratory system model over time, different personalized aerosol inhalation profiles captured by the aerosol generating device, can be fed back and applied to the respiratory system model, and the inhaled aerosol deposition for each aerosol inhalation profile can be determined in the region of interest, such that the model is enabled to determine an optimum aerosol inhalation profile which results in a target aerosol deposition over time in the region of interest of the respiratory system of the user. Specifically, the modular respiratory system model is able to determine transient aerosol dosimetry in different regions of interests of the respiratory system, comprising different airway generations. In addition, the disclosed modular respiratory system model reduces modelling work and execution time compared to existing modelling techniques.

Each airway module may comprise one or more component modules, wherein each component module represents a component of the respiratory system and comprises a port to input flow and a port to output flow.

Accordingly, the airway module represents at least a part of an airway generation and comprises a component of the respiratory system. Thus, the component of the respiratory system may be a component of an airway generation of the respiratory system. Such a component module may represent, for instance, the trachea, the bifurcation, the branch, the bronchium, or the bronchiole.

A number of airway modules of the plurality of airway modules may comprise a respective alveola module, each alveola module representing a number of alveoli of the respective airway module or generation, wherein each alveola module may comprise a port to receive flow.

For example, the number of airway modules of the plurality of airway modules which may comprise respective alveola modules, may represent deeper airway generations of the respiratory system. This provides for a detailed and comprehensive modelling of the respiratory system, enabling therewith a more realistic representation of the lungs. This also allows for an accurate reproduction of aerosol behavior and deposition in the deeper alveolar lung of the respiratory system.

The respiratory system model may be configured to determine, for each aerosol inhalation profile of a number of aerosol inhalation profiles, a corresponding deposition of the inhaled aerosol over time in each airway module of a region of interest of the respiratory system, for determining an optimal aerosol inhalation profile corresponding to a target aerosol deposition.

The model may be further configured to generate inhalation guidance instructions based on the optimal aerosol inhalation profile.

By determining an optimal aerosol inhalation profile, and generating inhalation guidance instructions based on the optimal aerosol inhalation profile, the inhalation behavior of a user may thereby be adapted to match the optimal aerosol inhalation profile corresponding to a desired target aerosol deposition. The inhalation guidance instructions may, for example, indicate to the user a personalized optimal aerosol inhalation profile, an inhalation-exhalation flow rate, or a puff rate, to interactively guide the user to adapt the inhalation behavior in order to reach the desired target aerosol deposition.

According to another aspect of the present invention, there is provided a system comprising an aerosol generating device and a settings determination unit, the system comprising: one or more processors configured to perform the steps of: determining, at the settings determination unit, one or more settings of the aerosol generating device based on a respiratory system model; and controlling the aerosol generating device to provide aerosol based on the one or more settings determined by the settings determination unit.

The one or more processors may be configured to perform the further steps of: obtaining, at the settings determination unit, data related to the aerosol generating device; and adapting the respiratory system model based on the data related to the aerosol generating device.

Adapting the respiratory system model may comprise at least one of: adapting aerosol data input to the respiratory system model based on aerosol data associated with the device; adapting aerosol data of the respiratory system model based on the settings of the device; adapting aerosol inhalation profiles based on aerosol inhalation profiles associated with the device or associated with one or more users of the device; and adapting a target aerosol deposition based on a target aerosol deposition associated with the device or with one or more users of the device.

Accordingly, by obtaining individualized data related to the aerosol generating such as aerosol data associated with the device, settings of the device, aerosol inhalation profiles and target aerosol deposition associated with the device or associated with one or more users of the device, this data may be used by the respiratory system model to adapt to the aerosol generating device.

Adapting the aerosol data of the respiratory system model based on the settings of the device may comprise at least one of: adjusting a frequency of vibration of a mesh of the heating system of the device to determine, at the settings determination unit, one or more adjusted frequencies of vibration; and adjusting a temperature of the mesh of the heating system of the aerosol generating device to determine, at the settings determination unit, one or more adjusted temperatures of the mesh.

Controlling the aerosol-generating device to provide aerosol based on the one or more settings determined by the settings determination unit may comprise using at least one of the one or more adjusted frequencies of vibration of the mesh and the one or more adjusted temperatures of the mesh.

Controlling the aerosol-generating device to provide aerosol based on the one or more settings determined by the settings determination unit may comprise adjusting at least one of a voltage and a current applied to a heating system of the aerosol generating device; adjusting at least one of a duration of power supplied to the heating system of the aerosol generating device; adjusting at least one of a pulse width and a pulse density of the power supplied to the heating system of the aerosol generating device; adjusting a rate of aerosol production of the aerosol generating device; and adjusting a volume of aerosol produced per puff from the aerosol generating device.

For example, the settings determination unit may automatically adjust the frequency of vibration of the mesh of the heating system of the device, such that a new frequency of vibration is determined which corresponds to a new aerosol concentration or dose. The settings determination unit may then adapt the aerosol data of the respiratory system model based on the new aerosol concentration or dose, hence ensuring that the model is fed with updated aerosol data corresponding to the new aerosol concentration or dose, to be able to determine a more accurate aerosol deposition in a specific region of interest, target lung depth level, or airway generation of the respiratory system model corresponding to the user of the device.

For example, the settings determination unit may automatically adjust the temperature of the mesh of the heating system of the device, such that a new temperature of the mesh is determined which corresponds to a new aerosol concentration or dose. The settings determination unit may then adapt the aerosol data of the respiratory system model based on the new aerosol concentration or dose, hence ensuring that the model is fed with updated aerosol data corresponding to the new aerosol concentration or dose, to be able to determine a more accurate aerosol deposition in a specific region of interest, target lung depth level, or airway generation of the respiratory system model corresponding to the user of the device.

The respiratory system model may be a modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises the captured aerosol inhalation profile, wherein each airway module is configured to determine, based on the input flow set by the inhalation topology module, deposition of the inhaled aerosol over time in the at least a part of the airway generation of the respiratory system represented by the airway module.

According to another aspect of the present invention, there is provided a computer-implemented modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises an aerosol inhalation profile, wherein each airway module is configured to determine, based on the input flow set by the inhalation topology module, deposition of the inhaled aerosol over time in the at least a part of the airway generation of the respiratory system represented by the airway module, such that the model is enabled to determine deposition of the inhaled aerosol over time in a region of interest of the respiratory system.

According to another aspect of the present invention, there is provided a computer-implemented method of generating a modular respiratory system model for transient aerosol dosimetry, the method comprising: obtaining physiological data related to a respiratory system; obtaining aerosol data related to aerosol properties; generating, based on the physiological data and the aerosol data, a plurality of airway modules, each airway module representing at least a part of an airway generation of the respiratory system; generating an inhalation topology module for setting an input flow to the respiratory system model over time, wherein the input flow comprises an aerosol inhalation profile; and generating the modular respiratory system model based on combining the plurality of airway modules and the inhalation topology module.

As used herein, the term “aerosol-generating device” refers to a device that interacts with an aerosol-forming substrate to generate an aerosol. An aerosol-generating device may interact with one or both of an aerosol-generating article comprising an aerosol-forming substrate, and a cartridge comprising an aerosol-forming substrate. In some examples, the aerosol-generating device may heat the aerosol-forming substrate to facilitate release of volatile compounds from the substrate. An electrically operated aerosol-generating device may comprise an atomizer, such as an electric heater, to heat the aerosol-forming substrate to form an aerosol.

As used herein, the term “aerosol-forming substrate” refers to a substrate capable of releasing volatile compounds that can form an aerosol. The volatile compounds may be released by heating or combusting the aerosol-forming substrate. As an alternative to heating or combustion, in some cases, volatile compounds may be released by a chemical reaction or by a mechanical stimulus, such as ultrasound. The aerosol-forming substrate may be solid or liquid or may comprise both solid and liquid components. An aerosol-forming substrate may be part of an aerosol-generating article.

The power supply may comprise control electronics. The control electronics may comprise a microcontroller. The microcontroller is preferably a programmable microcontroller. The electric circuitry may comprise further electronic components. The electric circuitry may be configured to regulate a supply of power to the heater assembly. Power may be supplied to the heater assembly continuously following activation of the system or may be supplied intermittently, such as on a puff-by-puff basis. The power may be supplied to the heater assembly in the form of pulses of electrical current.

The invention is defined in the claims. However, below there is provided a non-exhaustive list of non-limiting examples. Any one or more of the features of these examples may be combined with any one or more features of another example, embodiment, or aspect described herein.

Example Ex1: A computer-implemented modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises an aerosol inhalation profile, wherein each airway module is configured to determine, based on the input flow set by the inhalation topology module, deposition of the inhaled aerosol over time in the at least a part of the airway generation of the respiratory system represented by the airway module, such that the model is enabled to determine deposition of the inhaled aerosol over time in a region of interest of the respiratory system.

Example Ex2: The model according to example Ex1, wherein each airway module comprises one or more component modules, wherein each component module represents a component of the respiratory system and comprises a port to input flow and a port to output flow.

Example Ex3: The model according to example Ex2, wherein the component of the respiratory system comprises a trachea, a bifurcation, a branch, a bronchium, or a bronchiole.

Example Ex4: The model according to any of examples Ex2 and Ex3, wherein the component module further comprises a volume port configured to regulate a volume expansion of the component module.

Example Ex5: The model according to any of examples Ex2 and Ex4, wherein the component module further comprises a thermal port configured to regulate heat transfer in the component module.

Example Ex6: The model according to any of examples Ex2 to Ex5, wherein an airway module further comprises an input pressure drop element configured to apply a pressure drop on the flow inputted by the port of the component module

Example Ex7: The model according to any of examples Ex2 to Ex6, wherein each airway module further comprises an output pressure drop element configured to apply pressure drop on the flow outputted by the port of the component module to determine an intermediate flow.

Example Ex8: The model according to any of examples Ex1 to Ex7, further comprising one or more extra-thoracic airway modules representing extra-thoracic airway components of the respiratory system.

Example Ex9: The model according to example Ex8, wherein an extra-thoracic airway component of the respiratory system comprises a mouth, a nose, a bifurcation, or a larynx.

Example Ex10: The model according to example Ex9, wherein a first extra-thoracic airway module of the one or more extra-thoracic airway modules represents the mouth, and a second extra-thoracic airway module of the one or more extra-thoracic airway modules represents the nose.

Example Ex11: The model according to example Ex10, wherein setting the input flow to the respiratory system model over time comprises setting the input flow to the first extra-thoracic airway module representing the mouth or to the second extra-thoracic airway module representing the nose, over time, or setting a first input flow to the first extra-thoracic airway module and a second input flow the second extra-thoracic airway module, over time, wherein the first input flow and the second input flow constitute the input flow.

Example Ex12: The model according to any of examples Ex1 to Ex11, wherein the model comprises a single-path configuration, wherein each airway module of the single-path configuration is configured to represent an entire airway generation.

Example Ex13: The model according to any of examples Ex1 to Ex12, wherein the model is based on a hybrid respiratory system model, wherein one or more airway modules are configured to represent only a part of an airway generation.

Example Ex14: The model according to example Ex13, wherein the respective parts of the airway generations represented by the airway modules correspond to lobes of a lung of the respiratory system.

Example Ex15: The model according to any of examples Ex8 to Ex14, further configured to combine the plurality of the airway modules and the one or more extra-thoracic airway modules, and to determine branching of the input flow set to the respiratory system model from the extra-thoracic airway modules into each airway module of the respiratory system model.

Example Ex16: The model according to example Ex15, wherein determining the branching of input flow in each airway module comprises dividing the flow inputted to the airway module by a branch number parameter of the airway module to determine a flow in the airway module.

Example Ex17: The model according to any one of examples Ex1 to Ex16, wherein a number of airway modules of the plurality of airway modules comprise a respective alveola module, each alveola module representing a number of alveoli of the respective airway module, wherein each alveola module comprises a port to receive flow.

Example Ex18: The model according to example Ex17, further configured to combine the alveola module with a component module in each airway module of the number of airway modules, wherein combining the alveola module with the component module comprises determining a branching of the flow into the alveola module.

Example Ex19: The model according to example Ex18, wherein determining the branching of the flow into the alveola module comprises multiplying an intermediate flow by a branch number parameter in the airway module, to determine a total intermediate flow in the airway module, and dividing the total intermediate flow in the airway module by an alveola number parameter of the airway module to determine the flow received by the port of the alveola module.

Example Ex20: The model according to any one of examples Ex17 to Ex19, wherein the alveola module further comprises a thermal port configured to regulate heat transfer in the alveola module.

Example Ex21: The model according to any one of examples Ex17 to Ex20, wherein the alveola module further comprises a volume port configured to regulate a volume expansion of the alveola module.

Example Ex22: The model according to example Ex21, wherein setting the input flow to the respiratory system model over time comprises setting a predetermined volume expansion over time at the volume port of the alveola module or at the volume port of any component of the airway module, wherein the predetermined volume expansion over time corresponds to a rate of change of the volume over time.

Example Ex23: The model according to any one of examples Ex1 to Ex22, further comprising an input module configured for receiving input data.

Example Ex24: The model according to example Ex23, wherein the input data comprises at least one of: associated with the respiratory system of a subject, a selection of a region of interest of the respiratory system, a target aerosol deposition to be delivered to one or more airway generations or a region of interest, a respective branch number parameter for an airway module, a respective alveola number parameter for an alveola module, aerosol data related to aerosol properties, and data related to a number of aerosol inhalation profiles.

Example Ex25: The model according to example Ex24, wherein the input physiological data related to the respiratory system are used to adapt one or more airway modules, wherein the physiological data comprise geometric properties of one or more components of the respiratory system, wherein the geometric properties comprise at least one of a branch length, a branch diameter, a bifurcation angle, an angle to gravity.

Example Ex26: The model according to any one of examples Ex1 to Ex25, further configured to determine, for each aerosol inhalation profile of a number of aerosol inhalation profiles, a corresponding deposition of the inhaled aerosol over time in each airway module of a region of interest of the respiratory system, for determining an optimal aerosol inhalation profile corresponding to a target aerosol deposition.

Example Ex27: The model according to example Ex26, further configured to generate inhalation guidance instructions based on the optimal aerosol inhalation profile.

Example Ex28: The model according to example Ex27, wherein the inhalation guidance instructions comprise at least one of the optimal aerosol inhalation profile, an inhalation-exhalation flow rate, a puff rate, and a rate of variation of lungs volume, wherein the inhalation-exhalation flow rate, the puff rate, and the rate of variation of lungs volume are based on the optimal aerosol inhalation profile.

Example Ex29: A computer-implemented method of generating a modular respiratory system model for transient aerosol dosimetry, the method comprising: obtaining physiological data related to a respiratory system; obtaining aerosol data related to aerosol properties; generating, based on the physiological data and the aerosol data, a plurality of airway modules, each airway module representing at least a part of an airway generation of the respiratory system; generating an inhalation topology module for setting an input flow to the respiratory system model over time, wherein the input flow comprises an aerosol inhalation profile; and generating the modular respiratory system model based on combining the plurality of airway modules and the inhalation topology module.

Example Ex30: The method according to example Ex29, further comprising setting, by the inhalation topology module, the input flow to the respiratory system model over time.

Example Ex31: The method according to example Ex30, further comprising determining, based on the set input flow, deposition of the inhaled aerosol over time in the at least a part of the airway generation of the respiratory system represented by a respective airway module.

Example Ex32: The method according to any of examples Ex29 to Ex31, wherein each airway module comprises one or more component modules, wherein each component module represents a component of the respiratory system and comprises a port to input flow and a port to output flow.

Example Ex33: The method according to example Ex32, wherein the component of the respiratory system comprises a trachea, a bifurcation, a branch, a bronchium, or a bronchiole.

Example Ex34: The method according to any of examples Ex32 and Ex33, wherein the component module further comprises a volume port configured to regulate a volume expansion of the component module.

Example Ex35: The method according to any of examples Ex32 to Ex34, wherein the component module further comprises a thermal port configured to regulate heat transfer in the component module.

Example Ex36: The method according to any of examples Ex32 to Ex35, wherein an airway module further comprises an input pressure drop element configured to apply a pressure drop on the flow inputted by the port of the component module.

Example Ex37: The method according to any of examples Ex32 to Ex36, wherein each airway module further comprises an output pressure drop element configured to apply a pressure drop on the flow outputted by the port of the component module to determine an intermediate flow.

Example Ex38: The method according to any of examples Ex29 to Ex37, further comprising one or more extra-thoracic airway modules representing one or more extra-thoracic airway components of the respiratory system.

Example Ex39: The method according to example Ex38, wherein an extra-thoracic airway component of the respiratory system comprises a mouth, a nose, a bifurcation, or a larynx.

Example Ex40: The method according to example Ex39, wherein a first extra-thoracic airway module of the one or more extra-thoracic airway modules represents the mouth, and a second extra-thoracic airway module of the one or more extra-thoracic airway modules represents the nose.

Example Ex41: The method according to example Ex40, wherein setting the input flow to the respiratory system model over time comprises setting the input flow to the first extra-thoracic airway module representing the mouth or to the second extra-thoracic airway module representing the nose, over time, or setting a first input flow to the first extra-thoracic airway module and a second input flow to the second extra-thoracic airway module, over time, wherein the first input flow and the second input flow constitute the input flow.

Example Ex42: The method according to any of examples Ex29 to Ex41, wherein the model comprises a single-path configuration, wherein each airway module of the single-path configuration is configured to represent an entire airway generation.

Example Ex43: The method according to any of examples Ex29 to Ex42, wherein the model is based on a hybrid respiratory system model, wherein one or more airway modules are configured to represent only a part of an airway generation.

Example Ex44: The method according to example Ex43, wherein the respective parts of the airway generations represented by the airway modules correspond to lobes of a lung of the respiratory system.

Example Ex45: The method according to any of examples Ex38 to Ex44, further configured to combine the plurality of the airway modules and the one or more extra-thoracic airway modules and to determine branching of the input flow set to the respiratory system model from the extra-thoracic airway modules into each airway module of the respiratory system model.

Example Ex46: The method according to example Ex45, wherein determining the branching of input flow in each airway module comprises dividing the flow inputted to the airway module by a branch number parameter of the airway module.

Example Ex47: The method according to any one of examples Ex29 to Ex46, wherein a number of airway modules of the plurality of airway modules comprise a respective alveola module, each alveola module representing a number of alveoli of the respective airway module, wherein each alveola module comprises a port to receive flow.

Example Ex48: The method according to example Ex47, further configured to combine the alveola module with a component module in each airway module of the number of airway modules, wherein combining the alveola module with the component module comprises determining a branching of the flow into the alveola module.

Example Ex49: The method according to example Ex48, wherein determining the branching of the flow into the alveola module comprises multiplying an intermediate flow by a branch number parameter in the airway module, to determine a total intermediate flow in the airway module, and dividing the total intermediate flow in the airway module by an alveola number parameter of the airway module to determine the flow received by the port of the alveola module.

Example Ex50: The method according to any of examples Ex47 to Ex49, wherein the alveola module further comprises a volume port configured to regulate a volume expansion of the alveola module.

Example Ex51: The method according to any of examples Ex47 to Ex50, wherein the alveola module further comprises a thermal port configured to regulate heat transfer in the alveola module.

Example Ex52: The method according to example Ex51, wherein setting the input flow to the respiratory system model over time comprises setting a predetermined volume expansion over time at the volume port of the alveola module or at the volume port of any component of the airway module, wherein the predetermined volume expansion over time corresponds to a rate of change of the volume over time.

Example Ex53: The method according to example Ex34, wherein setting the input flow to the respiratory system model over time comprises setting a predetermined volume expansion over time at the volume port of any component of the airway module, wherein the predetermined volume expansion over time corresponds to a rate of change of the volume over time.

Example Ex54: The method according to any of examples Ex29 to Ex53, further comprising obtaining at least one of: a selection of a region of interest of the respiratory system, a target aerosol deposition to be delivered to one or more airway generations or a region of interest, and data related to a number of aerosol inhalation profiles.

Example Ex55: The method according to any of examples Ex29 to Ex54, further configured to determine, for each aerosol inhalation profile of a number of aerosol inhalation profiles, a corresponding deposition of the inhaled aerosol over time in each airway module of a region of interest of the respiratory system, for determining an optimal aerosol inhalation profile corresponding to a target aerosol deposition.

Example Ex56: The method according to example Ex55, further comprising generating an inhalation guidance instructions based on the optimal aerosol inhalation profile.

Example Ex57: The method according to example Ex56, wherein the inhalation guidance instructions comprise at least one of the optimal aerosol inhalation profile, an inhalation-exhalation flow rate, a puff rate, and a rate of variation of lungs volume, wherein the inhalation-exhalation flow rate, the puff rate, and the rate of variation of lungs volume are based on the optimal aerosol inhalation profile.

Example Ex58: A system comprising an aerosol generating device and an inhalation guidance unit, the system being configured for using the model of one of examples Ex1 to Ex28.

Example Ex59: A system comprising an aerosol generating device and an inhalation guidance unit, the system comprising a memory storing the model of one of examples Ex1 to Ex28.

Example Ex60: The system according to any of examples Ex58 and Ex59, comprising one or more processors configured to perform the steps of: capturing an aerosol inhalation profile of a user of the aerosol generating device; feeding the captured aerosol inhalation profile to the respiratory system model; generating, based on the respiratory system model, inhalation guidance instructions; and providing the inhalation guidance instructions to the user at the inhalation guidance unit.

Example Ex61: The system according to example Ex60, wherein capturing the aerosol inhalation profile of the user comprises determining, in real-time, at least one of an inhalation-exhalation flow rate, a puff rate, and a rate of variation of lungs volume.

Example Ex62: The system according to example Ex61, wherein determining the inhalation-exhalation flow rate is based on at least one of sensors counting inhalation-exhalation cycles or sensors capturing flow inhalation-exhalation flow profiles.

Example Ex63: The system according to example Ex61, wherein the aerosol generating device comprises sensors for counting aerosol puff cycles, wherein determining the puff rate is based on the counted aerosol puff cycles.

Example Ex64: A system comprising an aerosol generating device and a settings determination unit, the system being configured for using the model of one of examples Ex1 to

### Ex28.

Example Ex65: A system comprising an aerosol generating device and a settings determination unit, the system comprising a memory storing the model of one of examples Ex1 to Ex28.

Example Ex66: The system according to any of examples Ex64 and Ex65, comprising one or more processors configured to perform the steps of: determining, at the settings determination unit, one or more settings of the aerosol generating device based on the respiratory system model; controlling the aerosol-generating device to provide aerosol based on the one or more settings.

Example Ex67: The system according to example Ex66, wherein controlling the aerosol generating device to provide aerosol based on the one or more settings determined by the settings determination unit comprises at least one of: adjusting at least one of a voltage and a current applied to a heating system of the aerosol generating device; adjusting at least one of a duration of power supplied to the heating system of the aerosol generating device; adjusting at least one of a pulse width and a pulse density of the power supplied to the heating system of the aerosol generating device; adjusting a rate of aerosol production of the aerosol generating device; and adjusting a volume of aerosol produced per puff from the aerosol generating device.

Example Ex68: The system according to any of examples Ex66 and Ex67, wherein the wherein the one or more processors are configured to perform the further steps of: obtain data related to the aerosol generating device; and adapt the respiratory system model based on the data related to the aerosol generating device.

Example Ex69: The system according to example Ex68, wherein adapting the respiratory system model comprises at least one of: adapting aerosol data input to the respiratory system model based on aerosol data associated with the device; adapting aerosol data of the respiratory system model; adapting aerosol inhalation profiles of the inhalation topology module based on aerosol inhalation profiles associated with the device or associated with one or more users of the device; and adapting a target aerosol deposition based on a target aerosol deposition associated with the device or with one or more users of the device.

Example Ex70: The system according to example Ex69, wherein adapting the aerosol data of the respiratory system model comprises at least one of: adjusting a frequency of vibration of a mesh of the heating system of the device to determine one or more adjusted frequencies of vibration; and adjusting a temperature of the mesh of the heating system of the aerosol generating device to determine one or more adjusted temperatures of the mesh, wherein controlling the aerosol generating device to provide aerosol based on the one or more settings determined by the settings determination unit comprises using the determined at least one of: one or more adjusted frequencies of vibration and one or more adjusted temperatures of the mesh.

Example Ex71: A system comprising an aerosol generating device and an inhalation guidance unit, the system comprising: one or more processors configured to perform the steps of: capturing an aerosol inhalation profile of a user of the aerosol generating device; feeding the captured aerosol inhalation profile to a respiratory system model; generating, based on the respiratory system model, inhalation guidance instructions; and providing the inhalation guidance instructions to the user at the inhalation guidance unit.

Example Ex72: The system according to claim 71, wherein capturing the aerosol inhalation profile of the user comprises determining, in real-time, at least one of an inhalation-exhalation flow rate, a puff rate, and a rate of variation of lungs volume.

Example Ex73: The system according to examples Ex71 or Ex72, wherein the respiratory system model is a modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises the captured aerosol inhalation profile, wherein each airway module is configured to determine, based on the input flow set by the inhalation topology module, deposition of the inhaled aerosol over time in the at least a part of the airway generation of the respiratory system represented by the airway module.

Example Ex74: The system according to example Ex73, wherein each airway module comprises one or more component modules, wherein each component module represents a component of the respiratory system and comprises a port to input flow and a port to output flow.

Example Ex75: The system according to any one of examples Ex73 and Ex74, wherein a number of airway modules of the plurality of airway modules comprise a respective alveola module, each alveola module representing an alveola of the respective airway module, wherein each alveola module comprises a port to receive flow.

Example Ex76: The system according to any one of examples Ex73 to Ex75, wherein the respiratory system model is configured to determine, for each aerosol inhalation profile of a number of aerosol inhalation profiles, a corresponding deposition of the inhaled aerosol over time in each airway module of a region of interest of the respiratory system, for determining an optimal aerosol inhalation profile corresponding to a target aerosol deposition.

Example Ex77: The system according to example Ex76, wherein the respiratory system model is further configured to generate the inhalation guidance instructions based on the optimal aerosol inhalation profile.

Example Ex78: A system comprising an aerosol generating device and a settings determination unit, the system comprising: one or more processors configured to perform the steps of: determining, at the settings determination unit, one or more settings of the aerosol generating device based on a respiratory system model; and controlling the aerosol generating device to provide aerosol based on the one or more settings determined by the settings determination unit.

Example Ex79: The system according to example Ex78, wherein controlling the aerosol generating device to provide aerosol based on the one or more settings determined by the settings determination unit comprises at least one of: adjusting at least one of a voltage and a current applied to a heating system of the aerosol generating device; adjusting at least one of a duration of power supplied to the heating system of the aerosol generating device; adjusting at least one of a pulse width and a pulse density of the power supplied to the heating system of the aerosol generating device; adjusting a rate of aerosol production of the aerosol generating device; and adjusting a volume of aerosol produced per puff from the aerosol generating device.

Example Ex80: The system according to any of examples Ex78 and Ex79 wherein the one or more processors are configured to perform the further steps of: obtaining, at the settings determination unit, data related to the aerosol generating device; and adapting the respiratory system model based on the data related to the aerosol generating device.

Example Ex81: The system according to example Ex80, wherein adapting the respiratory system model comprises at least one of: adapting aerosol data input to the respiratory system model based on aerosol data associated with the device; adapting aerosol data of the respiratory system model; adapting aerosol inhalation profiles based on aerosol inhalation profiles associated with the device or associated with one or more users of the device; and adapting a target aerosol deposition based on a target aerosol deposition associated with the device or with one or more users of the device.

Example Ex82: The system according to example Ex81, wherein adapting the aerosol data of the respiratory system model comprises at least one of: adjusting a frequency of vibration of a mesh of the heating system of the device to determine, at the settings determination unit, one or more adjusted frequencies of vibration; and adjusting a temperature of the mesh of the heating system of the aerosol generating device to determine, at the settings determination unit, one or more adjusted temperatures of the mesh, wherein controlling the aerosol generating device to provide aerosol based on the one or more settings determined by the settings determination unit comprises using the determined at least one of: one or more adjusted frequencies of vibration and one or more adjusted temperatures of the mesh.

Example Ex83: The system according to one of examples Ex78 to Ex82, wherein the respiratory system model is a modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises the captured aerosol inhalation profile, wherein each airway module is configured to determine, based on the input flow set by the inhalation topology module, deposition of the inhaled aerosol over time in the at least a part of the airway generation of the respiratory system represented by the airway module.

Example Ex84: A computer-implemented modular respiratory system model for transient aerosol dosimetry, the model comprising: a plurality of airway modules, each airway module representing at least a part of an airway generation of the respiratory system; and an inhalation topology module configured for setting an input flow to the respiratory system model over time, wherein the input flow comprises an aerosol inhalation profile, wherein each airway module is configured to determine, based on the input flow set by the inhalation topology module, deposition of the inhaled aerosol over time in the at least a part of the airway generation of the respiratory system represented by the airway module, such that the model is enabled to determine deposition of the inhaled aerosol over time in a region of interest of the respiratory system.

Example Ex85: A computer-implemented method of generating a modular respiratory system model for transient aerosol dosimetry, the method comprising: obtaining physiological data related to a respiratory system; obtaining aerosol data related to aerosol properties; generating, based on the physiological data and the aerosol data, a plurality of airway modules, each airway module representing at least a part of an airway generation of the respiratory system; generating an inhalation topology module for setting an input flow to the respiratory system model over time, wherein the input flow comprises an aerosol inhalation profile; and generating the modular respiratory system model based on combining the plurality of airway modules and the inhalation topology module.

Examples will now be further described with reference to the figures in which:

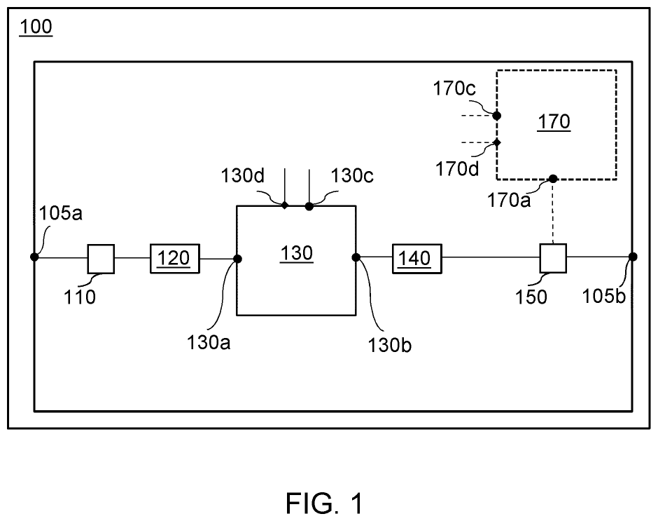

FIG. 1 shows an airway module associated with a modular respiratory system model;

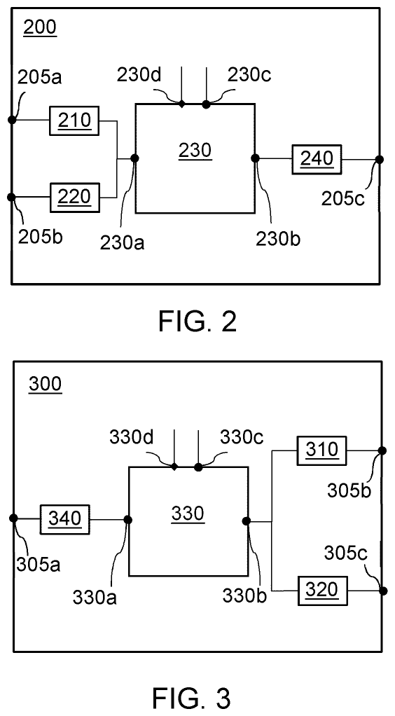

FIG. 2 shows a first embodiment of a bifurcation module representing a bifurcation in the respiratory system;

FIG. 3 shows a second embodiment of a bifurcation module representing a bifurcation in the respiratory system;

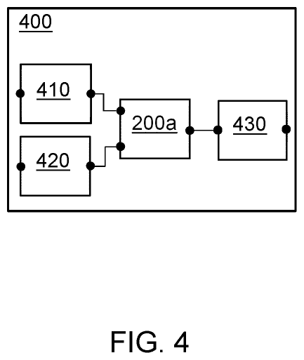

FIG. 4 shows an extra-thoracic combination module comprising extra-thoracic airway modules which represent extra-thoracic airway components of the respiratory system;

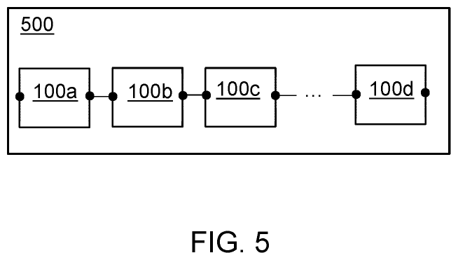

FIG. 5 illustrates a single-path configuration comprising a combination of airway modules associated with a single-path of the respiratory system model;

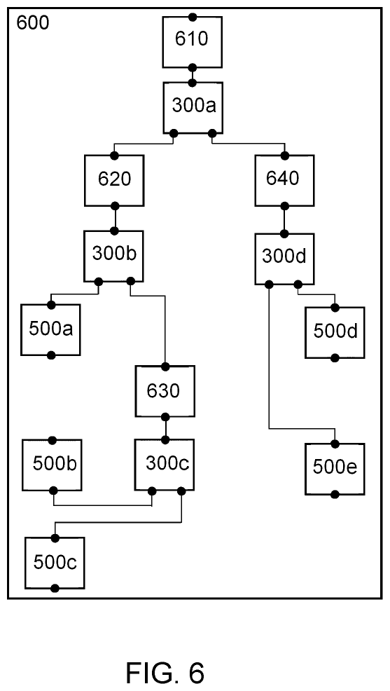

FIG. 6 illustrates a hybrid module comprising a combination of modules associated with a hybrid respiratory system model;

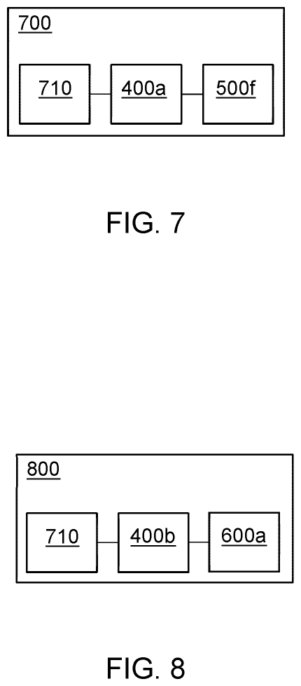

FIG. 7 illustrates a modular respiratory system model comprising a single-path configuration;

FIG. 8 illustrates a modular respiratory system model based on a hybrid respiratory system model;

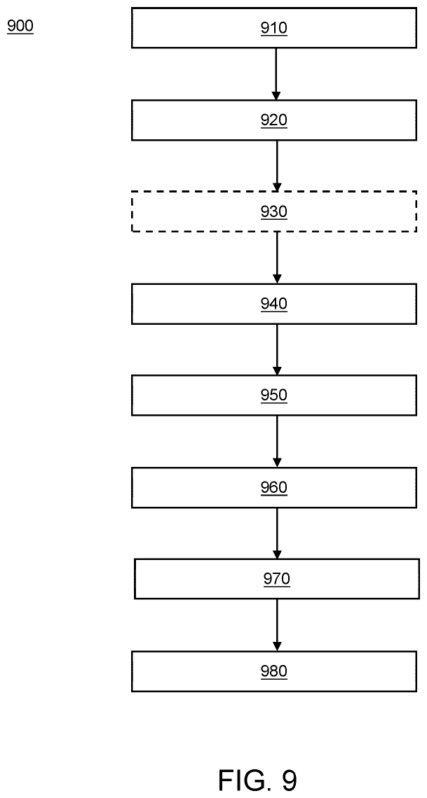

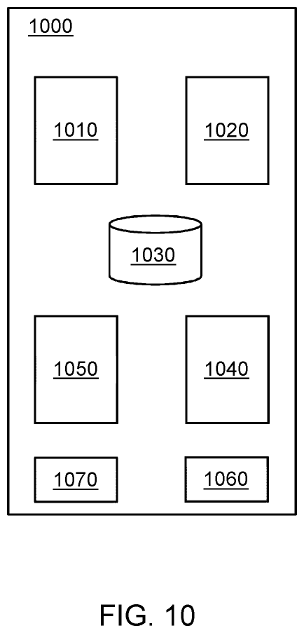

FIG. 9 is a flow diagram illustrating a computer-implemented method for generating a modular respiratory system model for transient aerosol dosimetry;

FIG. 10 illustrates a system comprising an aerosol generating device and an inhalation guidance unit;

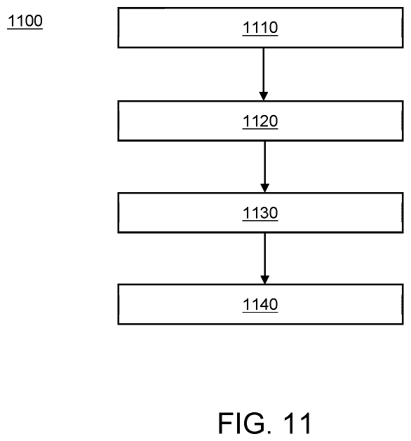

FIG. 11 is a flow diagram illustrating a computer-implemented method for providing inhalation guidance instructions to a user;

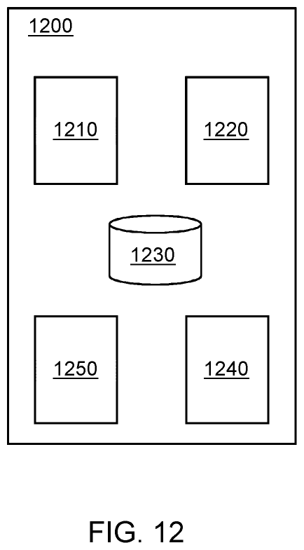

FIG. 12 illustrates a system comprising an aerosol generating device and a settings determination unit;

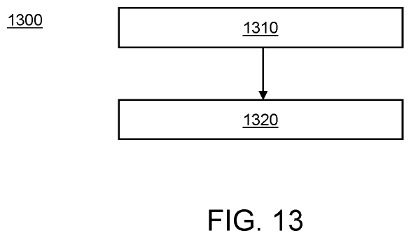

FIG. 13 is a flow diagram illustrating a computer-implemented method for controlling an aerosol generating device;

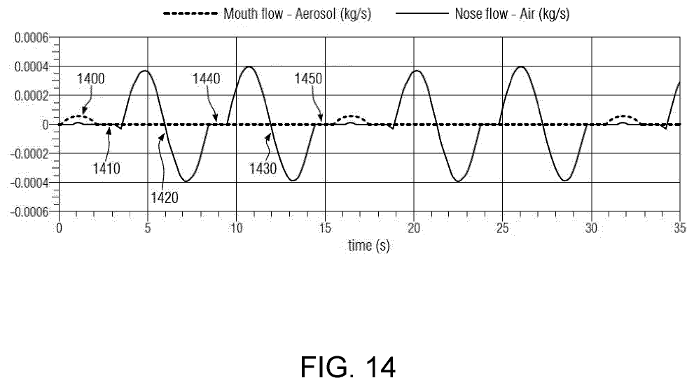

FIG. 14 illustrates an input flow comprising an aerosol inhalation profile; and

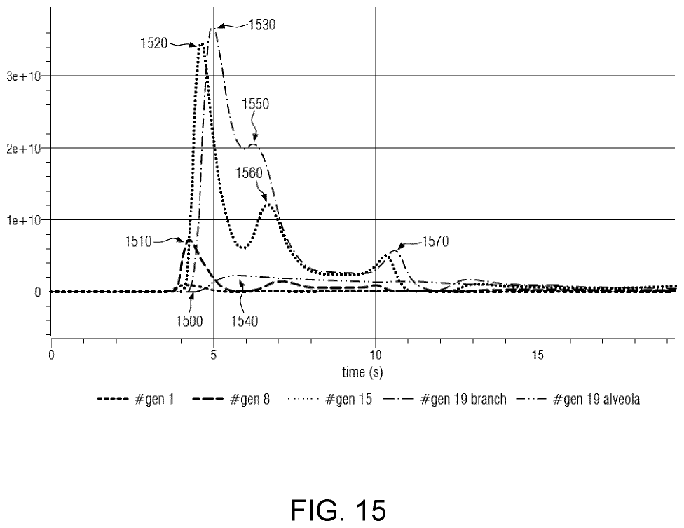

FIG. 15 illustrates results of transient aerosol deposition in different airway generations of a respiratory systems.

FIG. 1 illustrates an airway module 100 associated with a modular respiratory system model, and representing at least a part of an airway generation of the respiratory system. The airway module 100 comprises an input flow port 105a and output flow port 105b. The airway module 100 further comprises a component module 130 representing a component of the respiratory system and comprising a port 130a to input flow and a port 130b to output flow. The component of the respiratory system may comprise a trachea, a bifurcation, a branch, a bronchium, or a bronchiole.

The flow inputted by the input flow port 105a of the airway module 100 is divided by a branch number parameter of the airway module, using the element 110.

The branch number parameter is representative of the number of branches in the airway module. The branch number parameter of the airway module 100 may be based on physiological data related to the respiratory system. Accordingly, the airway module is representative of all similar components and all parallel branches in the at least a part of the airway generation of the respiratory system.

The physiological data related to the respiratory system may be used to adapt the airway module according to physiological data of a respiratory system of a subject. The subject may be a human or an animal. The physiological data may comprise geometric properties of one or more components of the respiratory system. The geometric properties may comprise at least one of a branch length, a branch diameter, a bifurcation angle, an angle to gravity.

The geometric properties of the one or more components may be based on at least one of a volume of expansion of lungs, a size of torso, an X-ray of the respiratory system, and a detailed airway tree morphometry. The geometric properties may be based on at least one of a height, an age, a gender, and a weight. The geometric properties may be based on generic geometric properties of the one or more components. The branch number parameter of the airway module is greater or equal to 1.

The airway module 100 further comprises an input pressure drop element 120 configured to apply a pressure drop on the flow inputted by the port 130a of the component module 130. The pressure drop in the airways of a respiratory system is directly related to the resistance of the airways of the respiratory system. The resistance of the airways is the opposition to flow caused by the forces of friction in the airways. The pressure drop element 120 is configured to model the resistance to the flow inputted by the port 130a of the component module 130.

The airway module 100 further comprises an output pressure drop element 140 configured to apply a pressure drop on the flow outputted by the port 130b of the component module 130 to determine an intermediate flow. The pressure drop element 140 is configured to model the resistance to the flow outputted by the port 130 b of the component module 130.

The modular respiratory system model is further configured to determine an output flow via port 105b of the airway module 100, by multiplying, using element 150, the intermediate flow by the branch number parameter of the airway module, to determine the output flow via port 105b of the airway module 100.

A number of airway modules representing deeper airway generations of the respiratory system further comprise an alveola module 170, represented by the dashed line square in FIG. 1. The alveola module 170 represents the cluster of alveoli of the airway module 100 and comprises a port 170a to receive flow. The modular respiratory system model may combine the alveola module 170 with the component module 130 in each airway module of the number of airway modules. Combining the alveola module 170 with the component module 130 comprises determining a branching of the flow into the alveola module 170. The alveola number parameter of the airway module may be based on the physiological data related to the respiratory system.

Determining the branching of the flow into the alveola module 170 comprises multiplying, by element 150, the intermediate flow by the alveola number parameter of the airway module 100 to determine the flow received by the port 170a of the alveola module 170.

When the airway module comprises an alveola module, the modular respiratory system model is further configured to determine an output flow via port 105b of the airway module 100 by multiplying, using element 150, the intermediate flow with a constant equals to the difference between the branch number parameter and the alveola number parameter, to determine the output flow via port 105b of the airway module 100.

The component module 130 further comprises a volume port 130c configured to regulate a volume expansion of the component module 130. The volume port 130c may be configured to set the input flow to the respiratory system model over time by setting a predetermined volume expansion over time at the volume port 130c of the component module 130 of the airway module 100. The predetermined volume expansion over time corresponds to a rate of change of the volume over time. The component module 130 further comprises one thermal port 130d configured to regulate heat transfer in the component module.

The alveola module 170 further comprises a volume port 170c configured to regulate a volume expansion of the alveola module. The volume port 170c may be configured to set the input flow to the respiratory system model over time by setting a predetermined volume expansion over time at the volume port 170c of the component module 130 of the airway module 100. The predetermined volume expansion over time corresponds to a rate of change of the volume over time. The alveola module 170 further comprises one thermal port 170d configured to regulate heat transfer in the alveola module.

The physical and chemical behavior of the aerosol may be represented by the publicly available AeroSolved model and its system implementation, AeroSolvedSystem (www.aerosolved.com, pmpsa-cfd GitHub). The well-known main processes and conservation laws are described in a fundamental element of AeroSolvedSystem, adapted to be used as either component modules, alveola modules, or extra-thoracic airway modules. Each one of these modules can be specified to represent a component of an individualized or personalized respiratory system by selecting the corresponding physiological data comprising personalized geometrical properties and by selecting the corresponding aerosol data related to aerosol properties.

The component module 130 may be a model of a component of the respiratory system. The alveola module 170 may be a model of a cluster of alveoli of the respiratory system. The component module 130, as well as the alveola module 170, may each be configured to impose an aerosol mixture mass conservation in time. The aerosol in the model can be in vapor or in liquid/solid phase. The aerosol mixture mass conservation in time defines an instantaneous rate of change of a total aerosol mixture mass in a volume of a vapor phase and of liquid/solid phases based on aerosol mixture mass fluxes at the volume ports of the model, and on source terms for liquid/solid and vapors through volume walls of the model, wherein the source terms for liquid/solid and vapors through the volume walls each model and the account for processes comprising deposition, condensation/evaporation, and absorption/desorption. This is illustrated by the following equation (1):

d/dt(Mₜₒₜ=wₐ+w_{b}−V W_{dep}−V W_{abs}, (1)

where Mₜₒₜ is the total aerosol mixture mass in the volume of both the vapor and of the liquid/solid phases. The variables wₐ and w_{b} are the aerosol mixture mass fluxes at the volume ports. Source terms for liquid or solid aerosols (W_{dep}) or vapors (W_{abs}) through the volume walls are included to account for processes comprising deposition, condensation/evaporation, and absorption/desorption.

The component module 130, as well as the alveola module 170, may each be configured to impose an aerosol mixture mass conservation in time for each aerosol species in vapor phase. The aerosol mixture mass conservation for each aerosol species in the vapor phase defines an instantaneous rate of change of a total aerosol mixture mass of the aerosol species in a volume of the vapor phase based on aerosol mixture mass fluxes at the volume ports of the model, and on source terms for vapors through volume walls of the model. The source terms for vapors through the volume walls of each model account for processes comprising deposition, condensation/evaporation, absorption/desorption, and nucleation. This is illustrated by the following equation (2):

(MₜₒₜY[i])=wₐYₐ[i]+wₚYₐ[i]+VΣⱼSₑᵥ[i,j]−VΣⱼS_{nuc}[i,j]−VS_{ab}[i], (2)

where mass balances are imposed for each species i in vapor phase Y[i].

The component module 130, as well as the alveola module 170, may each be configured to impose an aerosol mixture mass conservation in time for each aerosol species in liquid/sold phases, wherein the aerosol mixture mass conservation in time for each aerosol species in liquid/sold phases defines an instantaneous rate of change of a total aerosol mixture mass in a volume of the liquid/sold phases to be based on at least one of aerosol mixture mass fluxes at volume ports of each model, and on source terms for vapors through volume walls of the model, wherein the source terms for vapors through the volume walls of the model account for processes comprising deposition, condensation/evaporation, and nucleation. This is captured by the following equation (3):

d/dt(MₜₒₜZ[i,j])=wₐZ[i,j]+w_{b}Z_{b}[i,j]−V Sₑᵥ[i,j]+VS_{nuc}[i,j]−VS_{dep}[i,j], (3)

where mass balances are imposed for each species i in the liquid/solid phases Z [i,j] for each particle diameter j.

The component module 130, as well as the alveola module 170, may each be configured to impose a particle number density conservation in time for each particle size distribution associated with a diameter representing an evolving distribution of fixed shape. The particle number density conservation in time defines an instantaneous rate of change of a volume of the aerosol mixture to be based on at least one of aerosol mixture mass fluxes at the volume ports of the model, and on source terms for liquid/solid and vapors through volume walls of each model, wherein the source terms for liquid/solid and vapors through the volume walls of the model account for processes comprising deposition, condensation/evaporation, nucleation and coagulation. This is illustrated by the following equation (4):

d/dt(V N[j])=wₐNₐ[j]+w_{b}N_{b}[j]−V Jₑᵥ[j]+V J_{nuc}[j]−V J_{coag}[j]−V J_{dep}[j]. (4)

The processes comprising deposition, condensation/evaporation, and absorption/desorption are based on aerosol data related to the aerosol properties. The aerosol data related to the aerosol properties may comprise data related to one or more of material properties for aerosol species, distribution of aerosol droplets, and aerosol models.

The aerosol models may comprise Multiple Path Particle Dosimetry Model, MPPD, dosimetry models related to aerosol physics, and dosimetry models not directly related to aerosol physics. The dosimetry models not directly related to aerosol physics may comprise models describing mucociliary clearance.

In some embodiments, fully transient modelling of aerosol transport achieved by the model may further include modelling of chemical reactions. Accordingly, Equations (1)-(4) may be adapted to further include terms describing chemical reactions.

Accordingly, based on the above description comprising equations (1) to (4), full aerosol physics can be modelled. In addition, dynamic, fully transient modelling of aerosol transport, evolution and deposition in personalized whole-lung geometries can be achieved at an optimized computational cost. In embodiments, deposition with respect to aerosol droplets/particles is determined by modelling the aerosol strictly in the droplet/particle phase. In other embodiments, deposition with respect to gases/vapour is determined by modelling the aerosol in both the droplet/particle phase and in the gase/vapour phase including modelling the processes of absorption/desorption and condensation/evaporation, and is still referred to with the term deposition. FIG. 2 illustrates a bifurcation module 200 comprising a component module 230 representing a bifurcation. The component module comprises an input port 230a, an output port 230b, a volume port 230c, and a thermal port 230d. The bifurcation module further comprises two input ports 205a and 205b used to input flow, and one output port 205c used to output flow. The bifurcation module 200 further comprises pressure drop elements 210 and 220 used to apply pressure drop to the flow inputted by the input port 230a of the component module 200 via respective ports 205a and 205b of the bifurcation module. The bifurcation module 200 further comprises a pressure drop element 240 used to apply pressure drop to the flow outputted by the output port 230b of the component module. The properties of the bifurcation module 200, comprising the properties of the component module 230 and of the pressure drop elements 210, 220 and 240 are based on the physiological data.

FIG. 3 illustrates a bifurcation module 300 comprising a component module 330 representing a bifurcation. The component module comprises an input port 330a, an output port 330b, a volume port 330c, and a thermal port 330d. The bifurcation module further comprises one input port 305a used to input flow, and two output ports 305b and 305c used to output flow. The bifurcation module 300 further comprises a pressure drop element 340 used to apply pressure drop to the flow inputted by the input port 330a of the component module 300 via the input port 305a of the bifurcation module. The bifurcation module 300 further comprises pressure drop elements 310 and 320 used to apply pressure drop to the respective flow outputted by the output port 330b of the component module 300 which exits the bifurcation module via respective ports 305b and 305c. The properties of the bifurcation module 300, comprising the properties of the component module 330 and of the pressure drop elements 310, 320 and 340 are based on the physiological data.

FIG. 4 illustrates an extra-thoracic combination module 400 comprising a combination of extra-thoracic airway modules 410, 420, 200a, and 430 which may be configured to represent extra-thoracic airway components of the respiratory system. An extra-thoracic airway component of the respiratory system may be a mouth, a nose, a bifurcation, or a larynx. For example the extra-thoracic airway module 410 may be configured to represent a nose, the extra-thoracic airway module 420 may be configured to represent a mouth, and the extra-thoracic airway module 430 may be configured to represent a larynx. The extra-thoracic airway module 200a is based on the combination 200 of airway modules, and may be configured to represent a bifurcation used to combine the flow from the extra-thoracic airway module 410 representing the nose and the extra-thoracic airway module 420 representing the mouth.

FIG. 5 illustrates a single-path configuration module 500 comprising a combination of airway modules 100a to 100d associated with a single-path respiratory system model. Each airway module of the airway modules 100a to 100d is based on the airway module 100 of FIG. 1. Each airway module represents an entire airway generation corresponding to both a left lung and a right lung of the respiratory system. For example, the airway module 100a could be configured to represent the trachea or airway generation 1 of the respiratory system, the airway module 100b could be configured to represent a bronchial airway generation, airway generation 2, the airway module 100c could represent a bronchiolar airway generation, and the airway module 100d could be configured to represent an alveolar airway generation of the respiratory system. Airway module 100d may represent the alveolar generation as it includes the alveola module 170.

The single-path configuration module 500 presents a computationally efficient implementation of the respiratory system model, as the airway modules 100a to 100d are all based on the airway module 100. The model hence only includes a single flow path to be computed, proving for a computationally efficient way to determine aerosol deposition, evolution, absorption, and desorption, for any desired depth level of airway generations of the respiratory system.

According to another embodiment of the present invention, different sections of the respiratory system may be modelled in a detailed branch by branch manner. This detailed modelling may be expanded to deeper airway generations based on the availability of personalized data, the need for more detail, and the availability of computational power.

FIG. 6 illustrates a hybrid module comprising a combination 600 of airway modules 610 to 640, single-path configuration modules 500a to 500e, and bifurcation modules 300a to 300d, associated with a hybrid respiratory system model. Each airway module 610 to 640 may be based on the airway module 100 of FIG. 1. Each airway module represents a part of an airway generation, wherein each airway module may be configured to represent a part of an airway generation corresponding to a lobe of a lung of the respiratory system. In particular, each airway module may be configured to represent a part of the airway generation corresponding to a left lobe of one or more left lobes of a left lung of the respiratory system, or a part of the airway generation corresponding to a right lobe of one or more right lobes of a right lung of the respiratory system.

For example, the airway module 610 could be configured to represent the trachea or airway generation 1. The bifurcation module 300a could be configured to represent a bifurcation based on the bifurcation module 300 of FIG. 3. The airway module 620 could be configured to represent a branch of the airway generation corresponding to the right upper lobe of the right lung. The airway module 640 could be configured to represent a branch of the airway generation corresponding to the left upper lobe of the left lung of the respiratory system. The bifurcation modules 300b, 300c, and 300d could be configured to represent bifurcations each of which is based on the bifurcation module 300 of FIG. 3. The single-path configuration module 500a, which is connected to one port of the bifurcation module 300b, is based on the single-path configuration module 500 of FIG. 5. The module 500a may comprise a combination of airway modules which may be configured to represent the remaining parts of the right upper lobe of the right lung of the respiratory system. The remaining parts of the right upper lobe may comprise bronchial airway generations, bronchiolar airway generations, and alveolar airway generations.

The airway module 630 could be configured to represent a branch of the airway generation corresponding to the right middle lobe of the right lung of the respiratory system. The single-path configuration module 500b, which is connected to one port of the bifurcation module 300c, is based on the single-path respiratory system model and is similar to the module 500 of FIG. 5. The module 500b may comprise a combination of airway modules which may be configured to represent the remaining parts of the right middle lobe of the right lung of the respiratory system.

The remaining parts of the right middle lobe may comprise bronchial airway generations, bronchiolar airway generations, and alveolar airway generations.

The single-path configuration module 500c, which is connected to one port of the bifurcation module 300c, is based on the single-path configuration module 500 of FIG. 5. The module 500c may comprise a combination of airway modules which may be configured to represent the right lower lobe of the right lung of the respiratory system. The right lower lobe may comprise bronchial airway generations, bronchiolar airway generations, and alveolar airway generations.

The single-path configuration module 500d, which is connected to the airway module 300d, is based on the single-path configuration module 500 of FIG. 5. The module 500d may comprise a combination of airway modules which may be configured to represent the remaining parts of the left upper lobe of the left lung of the respiratory system. The remaining parts of the left upper lobe may comprise bronchial airway generations, bronchiolar airway generations, and alveolar airway generations.

The single-path configuration module 500e, which is connected to the one port of the bifurcation module 300d, is based on the single-path configuration module 500 of FIG. 5. The module 500e may comprise a combination of airway modules which may be configured to represent the left lower lobe of the left lung of the respiratory system. The left lower lobe may comprise bronchial airway generations, bronchiolar airway generations, and alveolar airway generations.

FIG. 7 illustrates a modular respiratory system model 700 based on a single-path configuration. The model 700 comprises an inhalation topology module 710 configured for setting an input flow to the respiratory system model 700 over time. The input flow may be set based on an aerosol inhalation profile. The model 700 further comprises an extra-thoracic combination module 400a, based on the extra-thoracic combination module 400, and comprising a combination of extra-thoracic airway modules which may be configured to represent extra-thoracic airway components of the respiratory system. The extra-thoracic components may include a nose, a mouth, a bifurcation, or a larynx. The model 700 further comprises a module 500f, based on the single-path configuration module 500 of FIG. 5, and comprising a combination of airway modules associated with the single-path respiratory system model. Each airway module of the combination of airway modules may be configured to represent at least a part of an airway generation of the respiratory system. Each airway module of combination of airway modules may be configured to determine, based on the input flow set by the inhalation topology module 710, deposition, evolution, absorption and desorption of the inhaled aerosol over time in the at least a part of the airway generation of the respiratory system represented by the airway module, such that the model 700 is enabled to determine deposition, evolution, absorption and desorption of the inhaled aerosol over time in a region of interest of the respiratory system.

Setting the input flow to the respiratory system model 700 over time may comprise setting an input flow to a first extra-thoracic airway module representing the mouth or to a second extra-thoracic airway module representing the nose, over time, or setting a first input flow to the first extra-thoracic airway module and a second input flow to the second extra-thoracic airway module, over time, wherein the first input flow and the second input flow constitute the input flow.

Setting the input flow to the respiratory system model 700 over time may further comprise setting the conditions of the input flow comprising setting at least one of the input flow topology and the input flow thermo-physical conditions. Setting the input flow thermo-physical conditions may comprise setting at least one of the input flow temperature and the input flow aerosol concentration properties.

The model 700 further comprises combining the plurality of the airway modules and the one or more extra-thoracic airway modules.

Combining the plurality of the airway modules may comprise determining branching of the input flow set to the model, by the inhalation topology module 710, from the extra-thoracic airway modules into each airway module of the respiratory system model. Branching the input flow may comprise setting the output flow from the output port 105b of an airway module to be equal to the input flow inputted by the input port 105a of a subsequent module.

The flow in the respiratory system model is a bidirectional flow, flowing in a first period in a forward direction along the flow path, and in a second period in a reverse direction along the flow path. The flow in the respiratory system model may flow from the extra-thoracic airway modules to the airway modules, or from the airway module to the extra-thoracic airway module, capturing a full inhalation-exhalation cycle.

Combining the plurality of the airway modules may comprise combining some or all of the thermal ports of the components modules in the region of interest of the respiratory system model. The combined thermal ports are configured to control the heat transfer in the region of interest of the respiratory system model. The modular respiratory system model 700 may further comprise an interface module to establish a closed loop interaction with external models.

FIG. 8 illustrates a modular respiratory system model 800 based on a hybrid respiratory system model. The model comprises an inhalation topology module 710 configured for setting an input flow to the respiratory system model 800 over time, wherein the input flow comprises an aerosol inhalation profile. The model further comprises an extra-thoracic combination module 400b, based on the extra-thoracic combination module 400, and comprising extra-thoracic airway modules which may be configured to represent extra-thoracic airway components of the respiratory system. The extra-thoracic components may include a nose, a mouth, a bifurcation, or a larynx.

The model further comprises a module 600a representing a hybrid module based on the hybrid module 600 of FIG. 6, and comprising a hybrid combination of airway modules associated with a hybrid respiratory system model. Each airway module is based on the airway module 100 of FIG. 1. Each airway module may be configured to represent a part of an airway generation corresponding to a lobe of a lung of the respiratory system, as explained above with reference to FIG. 6. Each airway module of the hybrid combination of airway modules may be configured to determine, based on the input flow set by the inhalation topology module 810, deposition or even deposition, evolution, absorption and desorption of the inhaled aerosol over time in the at least a part of the airway generation of the respiratory system represented by the airway module, such that the model 800 is enabled to determine deposition or even deposition, evolution, absorption and desorption of the inhaled aerosol over time in a region of interest of the respiratory system. Setting the input flow to the respiratory system model over time may comprise setting an input flow to the extra-thoracic airway module representing the mouth or the nose, over time.

The model 800 may further comprise combining the plurality of the airway modules and the one or more extra-thoracic airway modules. Combining the plurality of the airway modules may comprise determining branching of the input flow set to the model, by the inhalation topology module 710, from the extra-thoracic airway modules into each airway module of the respiratory system model. Branching the input flow may comprise setting the output flow from the output port 105b of an airway module: to be equal to the input flow inputted by the input port 105a of a subsequent module, or to be divided between the input ports of subsequent modules based on the physiological data related to respiratory system.

The flow in the respiratory system model can be a bidirectional flow. The flow in the respiratory system model may flow from the extra-thoracic airway modules to the airway modules, or from the airway module to the extra-thoracic airway module.

Combining the plurality of the airway modules may comprise combining some or all of the thermal ports of the components modules in the region of interest of the respiratory system model. The combined thermal ports are configured to control the heat transfer in the region of interest of the respiratory system model.

The modular respiratory system model 700, 800, may further comprise an interface module to establish a closed loop interaction with external models. For example, the respiratory system model 700, 800, which computes the aerosol dynamic evolution, transport and deposition inside a region of interest of the lung can be connected to physiological based models (PBPK) to predict the biological effect of the inhaled aerosol. The connection between the respiratory system model 700, 800, and the PBPK models may be performed with an interface element (not shown in FIGS. 7 and 8) which processes the output of the respiratory system model 700, 800, and computes the inputs for the PBPK. The interface element may also be used to include possible extra dosimetry models which are not directly related to the aerosol physics, for example, modelling mucociliary clearance.

The model 700 or 800 may be further configured to determine, for each aerosol inhalation profile of the number of aerosol inhalation profiles, a corresponding deposition of the inhaled aerosol over time in each airway module of the region of interest of the respiratory system, for determining an optimal aerosol inhalation profile corresponding to the target aerosol deposition. The model 700 or 800 may be further configured to generate inhalation guidance instructions based on the optimal aerosol inhalation profile. The inhalation guidance instructions comprise at least one of the optimal aerosol inhalation profile, an inhalation-exhalation flow rate, a puff rate, and a rate of variation of lungs volume, wherein the inhalation-exhalation flow rate, the puff rate, and the rate of variation of lungs volume are based on the optimal aerosol inhalation profile.

A system implementing the model 700 or 800 may be further configured to obtain data related to an aerosol generating device, and adapt the respiratory system model based on the data related to the aerosol generating device.

The data related to the aerosol generating device may comprise at least one of a type of the device, properties of the device, settings of the device, aerosol properties associated with the device, target aerosol deposition associated with the device or with one or more users of the device, and aerosol inhalation profiles associated with the device or with one or more users of the device. The type of the device is obtained from a list of devices comprising at least on of an inhaler and an electronic smoking device.

Adapting the respiratory system model may comprise at least one of: adapting the aerosol data of the respiratory system model based on the aerosol data associated with the device; adapting the aerosol data of the respiratory system model; adapting the aerosol inhalation profiles of the inhalation topology module based on aerosol inhalation profiles associated with the device or associated with one or more users of the device; and adapting the target aerosol deposition based on the target aerosol deposition associated with the device or with one or more users of the device.

Adapting the aerosol data of the respiratory system model may comprise adjusting a frequency of vibration of the mesh of the heating system of the device to determine one or more adjusted frequencies of vibration. Alternatively or additionally, adapting the aerosol data of the respiratory system model based on the settings of the device may comprise adjusting a temperature of the mesh of the heating system of the aerosol generating device to determine one or more adjusted temperatures of the mesh.

The model 700 or 800 may be further configured to output an instruction the aerosol generating device. The instruction may be sent to the control unit 1020 via the communication module 1030 of the aerosol generating device. The instruction may be checked and processed by the control unit 1020, and then executed by the designated components (e.g. the mesh of the heating system) of the aerosol generating device. The instruction may comprise at least one of the one or more adjusted frequencies of vibration, and the one or more adjusted temperatures of the mesh.

FIG. 9 is a flow diagram illustrating a computer-implemented method 800 of generating a modular respiratory system model 700, 800, for transient aerosol dosimetry. The modular respiratory system model 700, 800 enables a real-time modelling of the evolution of aerosol dosimetry over time for an individualized whole-lung geometry of a respirator system. The method begins at step 910 where physiological data related to a respiratory system is obtained. As described above, the physiological data related to the respiratory system may comprise geometric properties of one or more components of the respiratory system, wherein the geometric properties comprise at least one of a branch length, a branch diameter, a bifurcation angle, an angle to gravity.

At step 920, aerosol data related to aerosol properties are obtained. As described above the aerosol data related to the aerosol properties may comprise data related to one or more of material properties for aerosol species, distribution of aerosol droplets, and aerosol models.

At step 930, an optional selection of a region of interest of the respiratory system is obtained. The selection may comprise a target number of airway generations or a specific part of a lobe of the lung. In case no selection is obtained the model automatically selects a region of interest based on the physiological data.

At step 940, a plurality of airway modules, each representing at least a part of an airway generation of the respiratory system, are generated based on the physiological data and the aerosol data. As described above with respect to FIG. 1, each airway module comprises a component module 130 representing a component of the respiratory system and comprising a port 130a to input flow and a port 130b to output flow. The component of the respiratory system may comprise a trachea, a bifurcation, a branch, a bronchium, or a bronchiole. A number of airway modules representing deeper airway generations of the respiratory system further comprise an alveola module 170. The alveola module 170 comprises a port 170a to receive flow.

At step 950, an inhalation topology module for setting an input flow to the respiratory system model over time is generated. The input flow comprising an aerosol inhalation profile. The aerosol inhalation profile is based on the aerosol data.

At step 960, the modular respiratory system model is generated based on combining the plurality of airway modules and the inhalation topology module. The generated model may be based on a single-path respiratory system model, wherein each airway module is configured to represent an entire airway generation, as described above with respect to FIG. 7. The generated model may be based on a hybrid respiratory system model, wherein each airway module is configured to represent only a part of an airway generation corresponding to a lobe of a lung of the respiratory system, as described above with respect to FIG. 8.

At step 970, the input flow to the respiratory system model over time is set by the inhalation topology module. Setting the input flow to the respiratory system model over time may comprise setting an input flow to the extra-thoracic combined module 400, over time. The extra-thoracic combined module may comprise a first extra-thoracic airway module representing the nose and a second extra-thoracic airway module representing a mouth. Setting the input flow to the extra-thoracic combined module, over time, may comprise setting the input flow to the first extra-thoracic airway module representing the mouth or to the second extra-thoracic airway module representing the nose, over time, or setting a first part of the input flow to the first extra-thoracic airway module and a second part of the input flow to the second extra-thoracic airway module, over time, wherein the first input flow and the second input flow constitute the input flow.

Alternatively, a predetermined volume expansion over time may be set at the volume port of the alveola module or at the volume port of any component of the airway module, wherein the predetermined volume expansion over time corresponds to a rate of change of the volume over time. The flow in the respiratory system model is a bidirectional flow. The flow in the respiratory system model may flow from the extra-thoracic airway modules to the airway modules, or from the airway module to the extra-thoracic airway module.

At step 980, the deposition of the inhaled aerosol over time is determined, based on the set input flow, in the at least a part of the airway generation of the respiratory system represented by the airway module. A fully transient modelling of aerosol transport, evolution and deposition in personalized whole-lung geometries can be achieved by modelling the physical and chemical behavior of the inhaled aerosol from the set input flow, and by applying the related processes conservation laws, based on equations (1) to (4).

Hence, the model is enabled to determine deposition of the inhaled aerosol over time in any region of interest of the respiratory system. Determining the deposition of the inhaled aerosol over time in any region of interest may comprise determining the deposition of the inhaled aerosol over time in each airway generation of the respiratory system model or in a specific airway generation, or in specific part of a lobe of the lung.

Some or all of the method steps described above with regard to FIG. 9 may be implemented by a computer in that they are executed by (or using) a processor, a microprocessor, an electronic circuit or processing circuitry. For example, the implementation can be performed using a non-transitory storage medium such as a computer-readable storage medium. Such computer-readable media can be any available media that can be accessed by a general-purpose or special-purpose computer system.

Generally, the method can be implemented as a computer program product with a program code or computer-executable instructions, the program code or computer-executable instructions being operative for performing one of the methods when the computer program product runs on a computer. The program code or the computer-executable instructions may, for example, be stored on a computer-readable storage medium.

A storage medium (or a data carrier, or a computer-readable medium) may comprise, stored thereon, the computer program or the computer-executable instructions for performing one of the methods described herein when it is performed by a processor. An apparatus may comprise one or more processors and the storage medium mentioned above.

An apparatus may comprise means, for example processing circuitry like e.g. a processor communicating with a memory, the means being configured to, or adapted to, perform one of the methods described herein. A computer may have installed thereon the computer program or instructions for performing one of the methods described herein.

FIG. 10 illustrates a system 1000 comprising an aerosol generating device 1010 and an inhalation guidance unit 1020. The aerosol generating device may be configured for using the modular respiratory system model 700, 800. The system may further comprise a memory 1030 storing the modular respiratory system model 700, 800. The system 1000 may further comprise one or more processors 1040. The system may further comprise a communication module 1050 configured to send and receive instructions to/from other electronic modules or devices, or between the aerosol generating device 1010 and the inhalation guidance unit 1020. The system may further comprise sensors 1060 counting inhalation-exhalation cycles or sensors 1070 capturing inhalation-exhalation flow profiles.

In embodiments, the inhalation guidance unit 1020 may be internal to the aerosol generating device 1010, or may be external to the aerosol generating device 1010.

The inhalation guidance unit 1020 may provide inhalation guidance instructions to the user. The inhalation guidance instructions are generated based on the respiratory system model, and may be provided to the user using various actuators comprising displays, loudspeakers, and vibration actuators. The one or more processors 1040 may be configured to perform the following steps of the method 1100 to adjust the inhalation behavior of the user based on the provided inhalation guidance instructions, as illustrated in FIG. 11:
Step 1110: Capturing an aerosol inhalation profile of a user of the aerosol generating device;
Step 1120: Feeding the captured aerosol inhalation profile to the respiratory system model;
Step 1130: Generating, based on the respiratory system model, inhalation guidance instructions; and
Step 1140: Providing the inhalation guidance instructions to the user at the inhalation guidance unit.

Capturing the aerosol inhalation profile of the user may comprise determining, in real-time, at least one of an inhalation-exhalation flow rate, a puff rate, and a rate of variation of lungs volume. Determining the inhalation-exhalation flow rate may be based on sensors 1060 capturing inhalation-exhalation flow profiles. Determining the puff rate may be based on the aerosol puff cycles which may be counted by sensors 1050. Determining the rate of variation of lungs volume may be based on external sensors (not shown in FIG. 10) which may capture the expansion of the lungs volume using a chest belt, and which may send the determined rate to the aerosol generating device 1000 via the communication module 1030.

FIG. 12 illustrates a system 1200 comprising an aerosol generating device 1210 and a settings determination unit 1220. The aerosol generating device may be configured for using the modular respiratory system model 700, 800. The system may further comprise a memory 1230 storing the modular respiratory system model 700, 800. The system 1200 may further comprise one or more processors 1240. The system may further comprise a communication module 1250 configured to send and receive instructions to/from other electronic modules or devices, or between the aerosol generating device 1210 and the settings determination unit 1220. The system may further comprise sensors 1260 counting inhalation-exhalation cycles or sensors 1270 capturing inhalation-exhalation flow profiles, and may further comprise various actuators (not shown) for providing feedback or guidance to a user. In embodiments, the settings determination unit 1220 may be internal to the aerosol generating device 1210, or may be external to the aerosol generating device 1210.

The one or more processors 1240 may be configured to perform the following steps of the method 1300 to control the aerosol generating device 1210, as illustrated in FIG. 13:
Step 1310: Determining, at the settings determination unit 1220, one or more settings of the aerosol generating device 1210 based on the respiratory system model 700, 800; and
Step 1320: Controlling the aerosol generating device 1210 to provide aerosol based on the one or more settings determined by the settings determination unit 1220.

Controlling the aerosol generating device 1210 to provide aerosol based on the one or more settings determined by the settings determination unit 1220 may comprise at least one of: adjusting at least one of a voltage and a current applied to a heating system of the aerosol generating device; adjusting at least one of a duration of power supplied to the heating system of the aerosol generating device; adjusting at least one of a pulse width and a pulse density of the power supplied to the heating system of the aerosol generating device; adjusting a rate of aerosol production of the aerosol generating device; and adjusting a volume of aerosol produced per puff from the aerosol generating device.

Determining, at the settings determination unit 1220, one or more settings of the aerosol generating device 1210 based on the respiratory system model 700, 800, may comprise the steps of: obtaining, at the settings determination unit, data related to the aerosol generating device 1210; and adapting the respiratory system model 700, 800, based on the data related to the aerosol generating device 1210.

Adapting the respiratory system model 700, 800, may comprise at least one of: adapting aerosol data input to the respiratory system model 700, 800, based on aerosol data associated with the device 1210; adapting aerosol data of the respiratory system model 700, 800; adapting aerosol inhalation profiles of the inhalation topology module based on aerosol inhalation profiles associated with the device or associated with one or more users of the device; and adapting a target aerosol deposition based on a target aerosol deposition associated with the device or with one or more users of the device.

Adapting the aerosol data of the respiratory system model 700, 800 may comprise at least one of: adjusting a frequency of vibration of a mesh of the heating system of the device to determine, at the settings determination unit, one or more adjusted frequencies of vibration; and adjusting a temperature of the mesh of the heating system of the aerosol generating device to determine, at the settings determination unit, one or more adjusted temperatures of the mesh.

Controlling the aerosol generating device 1210 to provide aerosol based on the one or more settings determined by the settings determination unit 1220 may comprise using at least one of: the one or more adjusted frequencies of vibration and the one or more adjusted temperatures of the mesh.

In alternative embodiments, the setting determination unit 1220 of the system 1200 may be integrated within the system 1000 described above with respect to FIG. 10.

FIG. 14 illustrates an input flow set by the inhalation topology module to the respiratory system model over time. The input flow may be applied to the respiratory system model via extra-thoracic airway modules representing the nose and the mouth.

According to one embodiment of the present invention, the input flow may comprise a first input flow applied to a first extra-thoracic airway module, and a second input flow applied to a second extra-thoracic airway module. The first extra-thoracic airway module may be representing a nose, and the second extra-thoracic airway module may be representing a mouth. Alternatively, the first extra-thoracic airway module may be representing a mouth, and the second extra-thoracic airway module may be representing a nose. The first input flow may comprise an air inhalation profile, and the second input flow may comprise an aerosol inhalation profile. The first input flow and the second input flow constitute the input flow.

According to another embodiment of the present invention, setting the input flow to the respiratory system model over time may comprise setting the input flow to the first extra-thoracic airway module representing the mouth or to the second extra-thoracic airway module representing the nose, over time.

Setting the input flow to the respiratory system model over time may comprise setting the conditions of the input flow comprising setting at least one of the input flow topology and the input flow thermo-physical conditions. Setting the input flow thermo-physical conditions may comprise setting at least one of the input flow temperature and the input flow aerosol concentration properties.

As illustrated in the example of FIG. 14, during the first 2 seconds, an aerosol inhalation profile comprising an aerosol puff 1400 is applied to the extra-thoracic module representing a mouth. After a hold of 1 second 1410, an air inhalation profile comprising two full inhalation-exhalation cycles 1420, 1430 is applied to the extra-thoracic module representing the nose. After each inhalation cycle, a further hold of one second 1440, 1450 is applied. In this case, at the beginning of the inhalation-exhalation cycle 1420 the extra-thoracic airway module representing the mouth contracts and the aerosol puff is transferred to the airway modules representing the lung. Then the process is repeated by applying an aerosol puff and setting an input flow. According to one embodiment of the present invention, realistic and measured tabulated aerosol inhalation profiles and air inhalation profiles can be used by the inhalation topology module to set the input flow to the respiratory system model.

FIG. 15 illustrates results of transient aerosol deposition in different regions of interests of a modular respiratory system model based on a single-path configuration similar to the one shown in FIG. 7. In this case, a 4-region respiratory system model was simulated. An extra-thoracic region comprising extra-thoracic airway modules representing the nose, mouth, and larynx. A bronchiolar region comprising airway modules representing bronchial airway generations. A bronchiolar region comprising airway modules representing bronchiolar airway generations, and an alveolar region comprising airway modules representing alveolar airway generations of the respiratory system of a user.

FIG. 15 shows results of the particle number deposition profiles in different regions of interest. The results shown in FIG. 15 are obtained by using the 4-region respiratory system model described above, and by applying the input flow comprising the aerosol inhalation profile and the air flow profile visualized in FIG. 14. According to FIG. 15, aerosol starts to deposit in the trachea, i.e., airway generation 1, at around t=4 second 1500 when aerosol is transferred from the extra-thoracic airway module representing the mouth to the airway modules representing the lung. The respiratory system model captures the deposition peak moving in time deeper and deeper 1510, 1520, 1530 in the lung during the first inhalation 1420. At the end of the inhalation, aerosol reaches the alveolar region and the deposition in the alveoli starts 1540. Secondary peaks of deposition 1550, 1560 are seen during the first exhalation phase 1420. Finally, it is also possible to observe further depositions 1570 during the second inhalation-exhalation cycle 1430, due to the aerosol remaining in the lung volume.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1.-15. (canceled)

16. A system, comprising:
an aerosol-generating device;
an inhalation guidance unit; and
one or more processors configured to perform the steps of:
capturing an aerosol inhalation profile of a user of the aerosol-generating device,
feeding the captured aerosol inhalation profile to a respiratory system model,
generating, based on the respiratory system model, inhalation guidance instructions, and
providing the inhalation guidance instructions to the user at the inhalation guidance unit.

17. The system according to claim 16, wherein capturing the aerosol inhalation profile of the user comprises determining, in real-time, at least one of an inhalation-exhalation flow rate, a puff rate, and a rate of variation of lungs volume.

18. The system according to claim 16, wherein the respiratory system model is a modular respiratory system model for transient aerosol dosimetry, the respiratory system model comprising:
a plurality of airway modules, each airway module representing at least a part of an airway generation of the respiratory system, and
an inhalation topology module configured to set an input flow to the respiratory system model over time, wherein the input flow comprises the captured aerosol inhalation profile,
wherein each airway module is configured to determine, based on the input flow set by the inhalation topology module, deposition of the inhaled aerosol over time in the at least a part of the airway generation of the respiratory system represented by the airway module.

19. The system according to claim 18,
wherein each airway module comprises one or more component modules, and
wherein each component module represents a component of the respiratory system and comprises a port to input flow and a port to output flow.

20. The system according to claim 18,
wherein a number of airway modules of the plurality of airway modules comprise a respective alveola module, each alveola module representing an alveola of the respective airway module, and
wherein said each alveola module comprises a port to receive flow.

21. The system according to claim 18, wherein the respiratory system model is configured to determine, for each aerosol inhalation profile of a number of aerosol inhalation profiles, a corresponding deposition of the inhaled aerosol over time in each airway module of a region of interest of the respiratory system, for determining an optimal aerosol inhalation profile corresponding to a target aerosol deposition.

22. The system according to claim 21, wherein the respiratory system model is further configured to generate the inhalation guidance instructions based on the optimal aerosol inhalation profile.

23. A system, comprising:
an aerosol-generating device;
a settings determination unit; and
one or more processors configured to perform the steps of:
determining, at the settings determination unit, one or more settings of the aerosol-generating device based on a respiratory system model, and
controlling the aerosol-generating device to provide aerosol based on the one or more settings determined by the settings determination unit.

24. The system according to claim 23, wherein controlling the aerosol-generating device to provide aerosol based on the one or more settings determined by the settings determination unit comprises at least one of:
adjusting at least one of a voltage and a current applied to a heating system of the aerosol-generating device,
adjusting a duration of power supplied to the heating system of the aerosol-generating device,
adjusting at least one of a pulse width and a pulse density of the power supplied to the heating system of the aerosol-generating device,
adjusting a rate of aerosol production of the aerosol-generating device, and
adjusting a volume of aerosol produced per puff from the aerosol-generating device.

25. The system according to claim 23, wherein the one or more processors are further configured to perform the further steps of:
obtaining, at the settings determination unit, data related to the aerosol-generating device, and
adapting the respiratory system model based on the data related to the aerosol-generating device.

26. The system according to claim 25, wherein the adapting the respiratory system model comprises at least one of:
adapting aerosol data input to the respiratory system model based on aerosol data associated with the aerosol-generating device,
adapting aerosol data of the respiratory system model,
adapting aerosol inhalation profiles based on aerosol inhalation profiles associated with the device or associated with one or more users of the aerosol-generating device, and
adapting a target aerosol deposition based on a target aerosol deposition associated with the device or with one or more users of the aerosol-generating device.

27. The system according to claim 26, wherein the adapting the aerosol data of the respiratory system model comprises at least one of:
adjusting a frequency of vibration of a mesh of the heating system of the aerosol-generating device to determine, at the settings determination unit, one or more adjusted frequencies of vibration, and
adjusting a temperature of the mesh of the heating system of the aerosol-generating device to determine, at the settings determination unit, one or more adjusted temperatures of the mesh,
wherein the controlling the aerosol-generating device to provide aerosol based on the one or more settings determined by the settings determination unit comprises using the determined at least one of the one or more adjusted frequencies of vibration and the one or more adjusted temperatures of the mesh.

28. The system according to claim 23, wherein the respiratory system model is a modular respiratory system model for transient aerosol dosimetry, the respiratory system model comprising:
a plurality of airway modules, each airway module representing at least a part of an airway generation of the respiratory system, and
an inhalation topology module configured to set an input flow to the respiratory system model over time, wherein the input flow comprises the captured aerosol inhalation profile,
wherein said each airway module is configured to determine, based on the input flow set by the inhalation topology module, deposition of the inhaled aerosol over time in the at least a part of the airway generation of the respiratory system represented by the airway module.

29. A computer-implemented modular respiratory system model for transient aerosol dosimetry, the computer-implemented modular respiratory system model comprising:
a plurality of airway modules, each airway module representing at least a part of an airway generation of a respiratory system; and
an inhalation topology module configured for setting an input flow to the computer-implemented modular respiratory system model over time, wherein the input flow comprises an aerosol inhalation profile,
wherein said each airway module is configured to determine, based on the input flow set by the inhalation topology module, deposition of the inhaled aerosol over time in the at least a part of the airway generation of the respiratory system represented by the airway module, such that the computer-implemented modular respiratory system model is enabled to determine deposition of the inhaled aerosol over time in a region of interest of the respiratory system.

30. A computer-implemented method of generating a modular respiratory system model for transient aerosol dosimetry, the computer-implemented method comprising:
obtaining physiological data related to a respiratory system;
obtaining aerosol data related to aerosol properties;
generating, based on the physiological data and the aerosol data, a plurality of airway modules, each airway module representing at least a part of an airway generation of the respiratory system;
generating an inhalation topology module for setting an input flow to the respiratory system model over time, wherein the input flow comprises an aerosol inhalation profile; and
generating the modular respiratory system model based on combining the plurality of airway modules and the inhalation topology module.
